(19) United States
(12) Patent Application Publication
(10) Pub. No.: US 2009/0252702 A1
(43) Pub. Date: Oct. 8, 2009

(54) POLYMER-BASED ANTI-CANCER AGENTS

(75) Inventors: Lars Bruce, Viken (SE); Ingrid Bruce, Viken (SE)

Correspondence Address: YOUNG & THOMPSON, 209 Madison Street, Suite 500, ALEXANDRIA, VA, 22314, US

(73) Assignee: BRUCE MEDICAL AB, Viken (SE)

(21) Appl. No.: 12/298,437

(22) PCT Filed: Apr. 23, 2007
(86) PCT No.: PCT/SE07/00392

§ 371 Date: Oct. 24, 2008

Related U.S. Application Data
(60) Provisional application No. 60/794,089, filed on Apr. 24, 2006.

(51) Int. Cl. A61K 31/765; C08G 59/14; C12N 5/00; A61P 35/00

(52) U.S. Cl. 424/78.38; 525/523; 435/375

(57) ABSTRACT

The present invention relates to the use of amphiphilic block copolymers for treating and preventing cancer, and in particular by reducing the proliferation rate of cancer cells. Preferred block copolymers comprises a central hydrophobic chain, preferably a polypropylene oxide chain, two which at least two hydrophilic side chains, preferably polyethylene oxide chains, are connected.

### TECHNICAL FIELD

The present invention generally relates to cancer treatment, and in particular to the use of polymer-based anti-cancer agents in such cancer treatment.

### BACKGROUND

Cancer is a class of diseases characterized by uncontrolled division of cells and the ability of these cells to spread, either by direct growth into adjacent tissue through invasion, or by implantation into distant sites within the body by metastasis.

Today, cancer is a leading cause of death in humans and the number of affected individuals increases for each year. Although the different methods of treatment for cancer, e.g. chemotherapy, endocrine therapy, radiotherapy and surgery, have improved tremendously the last decades, they are far from perfect in terms of outcome for different cancer types. In addition, several of the known cancer treatments are marred by disadvantages in high treatment costs, side effects and patient suffering and relative inefficiency. For these reasons, extensive research is conducted to find alternative or complementary forms of cancer treatment.

Document [1] discloses the use of non-fermented osmotic laxative as active agents for the preparation of a medicinal product for treating colon and/or rectum cancers. An example of such a laxative is PLURONIC® F68 available from BASF Corporation. These compounds have laxative and gelling properties. The compounds attract and retain water inside the colon due to their physical-chemical properties, and are able to increase fecal excretion without fibers. It is believed that the laxative, non-fermented, osmotic and water-retaining properties of the compounds have a protective effect in relation to the two specific cancer types, colon and rectum cancer.

Document [2] discusses the ability of circulating tumor cells to develop into metastasis, where this ability is based on an inherent physiochemical adherence to the endothelium and the formation of a microclot. It is described that substances interfering with the coagulation process could be used in the prevention of tumor metastasis. Suggested substances include heparin, sodium warfarin and PLURONIC® F68. These substances can be used in connection with surgery to prevent metastasis secondary to operative tumor manipulation.

### SUMMARY

The present invention overcomes these and other drawbacks of the prior art arrangements.

It is a general object of the present invention to provide a polymer-based medicament that can be used for cancer treatment or prevention.

It is another object of the invention to provide polymer-based medicament that reduces the proliferation rate of cancer cells.

These and other objects are met by the invention as defined by the accompanying patent claims.

Briefly, the present invention involves usage of the unexpected anti-cancer effect of amphiphilic block copolymers. These copolymers are effective chemotherapeutic agents against a diversity of cancer types and have a proliferation rate reducing effect in the cancer cells.

The amphiphilic block copolymers of the present invention preferably comprise one hydrophobic polymer chain connected to at least two hydrophilic side chains. The hydrophobic polymer chain is preferably a central chain having a first end connected to at least one, preferably one or two, hydrophilic side chain and having a second end connected to at least one, preferably one or two, hydrophilic side chain.

Preferred amphiphilic block copolymers are those having the structure (I):

HO—(CH₂CH₂O)ₙ—(CH₂CHCH₃O)ₘ—(CH₂CH₂O)ₚ—H (I)

Thus, copolymers with a central polymer chain of propylene oxide flanked by side chains of ethylene oxide are preferred. In addition, n is preferably equal to p. Such copolymers are available under the trade name PLURONIC® by BASF Corporation.

Preferred such PLURONIC® copolymers of the invention are those that have an average ethylene oxide content of at least 40% w/w and preferably an average ethylene oxide content lower below 80% w/w. The average propylene oxide content of the amphiphilic block copolymer is preferably at least 2 000 g/mol, more preferably at least 3 000 g/mol, such as about 4 000±500 g/mol. An example of a preferred copolymer is PLURONIC® F127 having an average molecule weight of 12 600 g/mol, an average ethylene oxide content of 73.2±1.7% and a melting point of 56° C.

The inventors have surprisingly discovered that these copolymers have anti-cancer effect in terms of reducing or inhibiting the cell proliferation or growth rate of cancer cells and the reducing the DNA synthesis of cancer cells. This surprising effect may at least partly be due to the effect of the copolymers in binding to cell membranes and blocking the binding of different growth factors to their respective receptors on the membrane.

The pharmaceutical composition of the invention preferably comprises a single amphiphilic block copolymer of the invention or a mixture of at least two such copolymers as the sole chemotherapeutic agents.

### SHORT DESCRIPTION OF THE DRAWINGS

The invention together with further objects and advantages thereof, may best be understood by making reference to the following description taken together with the accompanying drawings, in which:

FIG. 1 is a diagram illustrating the effects of PLURONIC® F127 on the growth rate of human breast cancer cell line MCF-7;

FIG. 2 is a diagram illustrating the effects of PLURONIC® F127 on the growth rate of human breast cancer cell line SK-BR-3;

FIG. 3 is a diagram illustrating the effects of PLURONIC® F127 on the growth rate of FCS-stimulated human vascular smooth muscle cells;

FIG. 4 is a diagram illustrating the effects of PLURONIC® F127 on the growth rate of unstimulated and FCS-stimulated rat aortic smooth muscle cells;

FIG. 5 is a diagram illustrating a comparison of cell mediated cytotoxicity of PLURONIC® F127 and Triton X-100;

FIG. 6 is a diagram illustrating relative cell growth inhibiting effect of different amphiphilic block copolymers on FCS-stimulated human vascular smooth muscle cells;

FIG. 7 is a diagram illustrating the correlation between growth rate inhibition of PLURONIC® F127 on stimulated rat aortic muscle cells and the effect of PLURONIC® F127 in blocking fibroblast growth factor binding to receptors on the smooth muscle cells;

FIG. 8 is a diagram the effect of PLURONIC® F127 in blocking platelet derived growth factor binding to receptors on rat aortic smooth muscle cells;

FIG. 9 is a diagram illustrating cell density in a hollow fiber with U937/gtb cancer cells implanted in mice with or without treatment with PLURONIC® P105;

FIG. 10 is a diagram illustrating cell density in a hollow fiber with H69 cancer cells implanted in mice with or without treatment with PLURONIC® P105;

FIG. 11 is a diagram illustrating survival index of U937/gtb cancer cells exposed to different amphiphilic block copolymers;

FIGS. 12A to 12O are diagrams illustrating survival index of U937/gtb cancer cells exposed to different amphiphilic block copolymers; and

FIGS. 13A to 13C are diagrams illustrating survival index of different cancer cell lines exposed to PLURONIC® P84, F127 or L121.

### DETAILED DESCRIPTION

The present invention generally relates to cancer treatment and in particular to the use of amphiphilic block copolymers for inhibiting and reducing the growth and proliferation rate of cancer cells

The active anti-cancer compounds of the present invention are amphiphilic block copolymers of hydrophobic and hydrophilic monomers. The block copolymers therefore comprise at least one water-soluble (hydrophilic) part and at least one less water-soluble or even water-insoluble (hydrophobic) part. In presently preferred block copolymers, a central hydrophobic chain is surrounded by at least two hydrophilic side chains. More preferably, the central hydrophobic chain has a first chain end connected to at least one, preferably one or two, hydrophilic side chains and has a second chain end connected to at least one, preferably one or two, hydrophilic side chains. Formula (II) and (III) below schematically illustrate such preferred amphiphilic block copolymers:

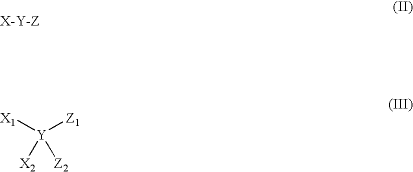

where X, X₁, X₂ and Z, Z₁, Z₂ represent a respective hydrophilic side chain and Y represents a hydrophobic central chain. In a preferred implementation X=Z, and X₁=X₂, Z₁=Z₂ and more preferably X₁=X₂=Z₁=Z₂.

In a preferred embodiment, the amphiphilic block copolymers of the present inventions are block copolymers of ethylene oxide and propylene oxide. Several different such copolymer are available today from different manufactures, including the polymers PLURONIC® and TETRONIC® from BASF Corporation.

Briefly, PLURONIC® is a copolymer of ethylene oxide (EO) and propylene oxide (PO) having the general structure (III):

(EO)ₙ-(PO)ₘ-(EO)ₚ (IV)

or the more detailed structure (I):

HO—(CH₂CH₂O)ₙ—(CH₂CHCH₃O)ₘ—(CH₂CH₂O)ₚ—H (I)

In a preferred embodiment n=p.

Table I below lists several PLURONIC® polymers available from BASF and that can be used according to the present invention.

TABLE I
PLURONIC ® copolymers

| | Average molecular | Viscosity | Solubility in H₂O | Melt point |
|---|---|---|---|---|
| Name | weight (g/mol) | (cps) | at 25° C. (%) | (° C.) |
| L31 | 1 100 | 175* | >10 | — |
| L35 | 1 900 | 375* | >10 | — |
| L43 | 1 850 | 310* | >10 | — |
| L44 | 2 200 | 440* | >10 | — |
| L61 | 2 000 | 325* | insoluble | — |
| L62 | 2 500 | 450* | >10 | — |
| L64 | 2 900 | 850* | >10 | — |
| L81 | 2 750 | 475* | insoluble | — |
| L92 | 3 650 | 700* | >1 | — |
| L101 | 3 800 | 800* | insoluble | — |
| L121 | 4 400 | 1 200* | insoluble | — |
| P65 | 3 400 | 180** | >10 | — |
| P84 | 4 200 | 280** | >10 | — |
| P85 | 4 600 | 310** | >10 | — |
| P103 | 4 950 | 285** | >10 | — |
| P104 | 5 900 | 390** | >10 | — |
| P105 | 6 500 | 750* | >10 | — |
| P123 | 5 750 | 350** | >10 | — |
| F38 | 4 700 | 260*** | >10 | 48 |
| F68 | 8 400 | 1 000*** | >10 | 52 |
| F77 | 6 600 | 480*** | >10 | 48 |
| F87 | 7 700 | 700*** | >10 | 49 |
| F88 | 11 400 | 2 300*** | >10 | 54 |
| F98 | 1 300 | 2 700*** | >10 | 58 |
| F108 | 14 600 | 2 800*** | >10 | 57 |
| F127 | 12 600 | 3 100*** | >10 | 56 |

*Viscosity [Brookfield] at 25° C.
**Viscosity [Brookfield] at 60° C.
***Viscosity [Brookfield] at 77° C.

As is known in the art, the alphabetical designation of the PLURONIC® product name denotes the physical form of the product at 25° C., where “L” represents liquid, “P” represents paste and “F” represents solid form. The first digit or the two first digits in a three-digit product name multiplied by 300 indicates the approximate molecular weight of the hydrophobe central polypropylene oxide chain. The last digit, when multiplied by 10, indicates the approximate ethylene oxide content (in %) of the polymer. This ethylene oxide content can be calculated from equation (1):

$$EO = \frac{44 ( n + p )}{44 ( n + p ) + 58 m} \qquad ( 1 )$$

where m, n and p are defined as in structure (I).

If the hydrophobic or lipid soluble part (PO) is reduced too much, i.e. m is a small integer number, the growth inhibitory effect is markedly reduced. As a consequence, preferred PLURONIC® copolymers of the present invention are therefore those that have a hydrophobic part that is at least about 2 000 g/mol, i.e. those PLURONIC® polymers of Table I that have a three-digit product name or where the first digit in the product name is larger than six.

In addition, PLURONIC® copolymers having a large hydrophilic content, i.e. an approximate ethylene oxide content of about 80% or more, have also been shown to have the least effective anti-cancer effect of the tested copolymers. These copolymers have an 8 as the last digit of the product name in Table I.

If the ethylene oxide content of the block copolymer is too low in relation to the propylene oxide content, the copolymer is less water soluble or even water insoluble. Such block copolymers may be less useful clinically as non-water based solvent then has to be used.

Furthermore, if both m, n and p in structure (I) are too low, such as L31, L42, L43, L44, L61, L62 and L63, i.e. a short relative hydrophobic block copolymer, the polymer becomes toxic for both cancerous and non-cancerous cells. As a consequence, lower pharmaceutical concentrations must be used for such copolymers.

Currently preferred examples of PLURONIC® copolymers include F127, P84, P105, P123, F87 and L121 and in particular F127.

Experiments have been conducted in which one of the hydrophilic side chains of a PLURONIC® copolymer is removed. In such a case, the inhibitory effect is markedly reduced or even lost. As a consequence, preferred amphiphilic copolymers of the present invention comprises at least two hydrophilic (polyethylene oxide) chains connected to a hydrophobic (polypropylene oxide) chain.

Other related copolymers that can be used according to the invention are TETRONIC® polymers also available from BASF Corporation. These copolymers can be represented by the following structure (V):

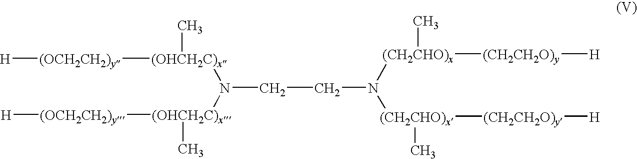

Table II below lists some properties of available TETRONIC® polymers.

TABLE II
TETRONIC ® copolymers

| | Average molecular | Viscosity | Solubility in H₂O | Melt point |
|---|---|---|---|---|
| Name | weight (g/mol) | (cps) | at 25° C. (%) | (° C.) |
| 304 | 1 650 | 450* | >10 | — |
| 701 | 3 600 | 600* | insoluble | — |
| 901 | 4 700 | 700* | insoluble | — |
| 904 | 6 700 | 320** | >10 | — |
| 908 | 25 000 | 325*** | >10 | 58 |
| 1107 | 15 000 | 1 100*** | >10 | 51 |
| 1301 | 6 800 | 1 000* | insoluble | — |
| 1307 | 18 000 | 2 700*** | >10 | 54 |

*Viscosity [Brookfield] at 25° C.
**Viscosity [Brookfield] at 60° C.
***Viscosity [Brookfield] at 77° C.

Of these TETRONIC® copolymers, TETRONIC® 1307 is a currently preferred amphiphilic copolymer according to the present invention. The 1307 copolymer has efficient anti-cancer effect, while being water soluble and relatively non-toxic to non-proliferating cells.

Also other amphipathic (amphiphilic) block copolymers can be used according to the invention. For example, a copolymer having a central polystyrene chain connected to respective side chains of polyethylene oxide has growth inhibitory effect. Thus, the present invention also encompassed other amphiphilic block copolymer besides those comprising a polyethylene oxide chain and multiple polyethylene oxide side chains.

BASF Corporation also has other related amphiphilic block copolymers that are related to the PLURONIC® and TETRONIC® copolymers. PLURONIC® R is a copolymer in which the ethylene oxide and the propylene oxide have be changed places as compared to PLURONIC®. In other words, the polymer has the following general structure:

HO—(CH₂CHCH₃O)ₖ—(CH₂CH₂O)_{q}—(CH₂CHCH₃O)ᵣ—H (VI)

TABLE III
PLURONIC ® R copolymers

| | Average molecular | Viscosity | Solubility in H₂O | Melt point |
|---|---|---|---|---|
| Name | weight (g/mol) | (cps) | at 25° C. (%) | (° C.) |
| 10R5 | 1 950 | 440* | >10 | — |
| 17R2 | 2 150 | 450* | >10 | — |
| 17R6 | 2 650 | 600* | >10 | — |
| 25R4 | 3 600 | 1 100* | >10 | — |
| 31R1 | 3 250 | 660* | >1 | — |

*Viscosity [Brookfield] at 25° C.

Correspondingly, copolymers in which the ethylene oxide and the propylene oxide of TETRONIC® have changed places are denoted TETRONIC® R polymers. Table IV lists such polymers available from BASF Corporation.

TABLE IV
TETRONIC ® R copolymers

| | Average molecular | Viscosity | Solubility in H₂O | Melt point |
|---|---|---|---|---|
| Name | weight (g/mol) | (cps) | at 25° C. (%) | (° C.) |
| 150R1 | 8 000 | 1 840* | insoluble | — |
| 90R4 | 6 900 | 3 870* | >10 | — |

*Viscosity [Brookfield] at 25° C.

It is to be noted that when molecular weight of the copolymers is stated in this document, there is meant the average theoretical molecular weight. As is well known to the person skilled in the art, in a given batch of a particular copolymer not all polymer molecules will have identical polymer length and molecular weight. Thus, a given molecular weight is an average value and there is a distribution around this average value. The same discussion of distribution around an average value applies to the hydrophilicity of the copolymer, e.g. as expressed by the average ethylene oxide content of the polymer.

The copolymers of the invention are effective inhibitors of cancer growth in vitro even at very low doses. The growth-inhibiting effect is furthermore more pronounced in rapidly growing cancer cells as compared to slowly growing cancer cells. In addition, at least some of the amphiphilic block polymers of the present invention do not have any cell proliferating affecting function on non-cancerous cells, unless they are stimulated by the addition of different growth factors.

The copolymers can be used to reduce and normalize the growth rate of different types of cancer cell lines. The copolymers furthermore seem to reduce the high proliferation down to the normal growth rate but not further. As a consequence, non-cancerous cells will not be affected since they already proliferate at the low normal growth rate.

Once the growth rate of the cancer cells has been diminished, the immune system of the (human) patient can more effectively handle and combat the cancer cells to eliminate the cancer.

The copolymers of the present invention may also have affect in preventing or at least reducing the rate at which mutation arises in the cancer cells. This finding is extremely important since it reduces the risk of forming cancer cells that, due to mutations, are more prone to avoid or combat the inherent cancer defense mechanisms of a subject.

In accordance with the invention, the amphiphilic block copolymers can be provided as pharmaceutically acceptable formulations using formulation methods known to those of ordinary skill in the art. These formulations can be administered by standard routes. In general, the copolymer may be administered intravenously, intraperitoneally, subcutaneously, buccally, rectally, dermally, nasally, orally, tracheally, bronchially, topically, by any other patenteral route or via inhalation, in the form of a pharmaceutical preparation comprising the active ingredient in a pharmaceutically acceptable dosage form.

A currently preferred administration route is an intravenous administration, in which the pharmaceutical medical composition comprises amphiphilic copolymer of the invention in a solution of a selected solvent.

In a particular administration implementation, the copolymer-containing solution is injected once or preferably at multiple time instants to a person in need of cancer treatment. It could also be possible to employ a continuous or semi-continuous supply of the medicament from e.g. a medical pump or other administration equipment. Also administrations through so-called slow-release is possible and within the scope of the present invention.

In another particular implementation, a local administration in or in connection with the tumor can be used to allow a relatively high local concentration of the active ingredient. This local administration can be accompanied by one or more systemic administrations.

In general, the formulations are prepared by uniformly and intimately bringing into associate the active ingredient with preferably liquid carriers or sometimes finely divided solid carriers or both, and then, if necessary, shaping the product.

Formulations suitable for parenteral administration include aqueous and non-aqueous sterile injection solutions which may contain anti-oxidants, buffers, bacteriostats and solutes which render the formulation isotonic with the blood of the intended recipient, and aqueous and non-aqueous sterile suspensions which may include suspending agents and thickening agents. The formulations may be presented in unit-dose or multi-dose containers, for example, sealed ampoules and vials, and may be stored in a freeze-dried (lyophilized) conditions requiring only the addition of the sterile liquid carrier, for example, water for injections, immediately prior to use.

In particular for water-insoluble copolymers of the present invention other media besides aqueous media can be used when injecting the pharmaceutics. An example of such a media is polyethylene glycol (PEG). Other examples include oil-in-water or water-in-oil emulsions. A mineral oil or other oily substance such as Drakeol 6VR or Drakeol 5 (Penreco, Butler, Pa.) can be used as the oil phase of the emulsion. The aqueous phase can be physiologic phosphate buffered saline or other physiologic salt solution. The ratio of oil to water is preferably between approximately 80:20 and 1:100.

Formulations of suitable for oral administration may be presented as capsules, cachets or tablets each containing a predetermined amount of the active ingredient, as a powder or granules; as a solution or a suspension or emulsion in an aqueous liquid or a non-aqueous liquid. Formulations suitable for topical administration to the skin may be presented as ointments, creams, gels and pastes comprising the ingredient to be administered in a pharmaceutical acceptable carrier. Formulations for rectal administration may be presented as a suppository with a suitable base comprising, for example, cocoa butter or a salicylate. Formulations suitable for vaginal administration may be presented as pessaries, tamports, creams, gels, pastes, foams or spray formulations containing in addition to the active ingredient such carriers as are known in the art to be appropriate.

Examples of unit dosage formulations are those containing a daily dose or unit, daily sub-dose, as herein above recited, or an appropriate fraction thereof, of the administered ingredient.

The maximum allowable dosage that can be used according to the present invention depends, among others, on the toxicity of the particular amphiphilic block copolymer, its anti-cancer effect, i.e. growth rate inhibitory effect, and the administration route. The maximum allowable concentration of an amphiphilic copolymer can be estimated according the toxicity study described in the Example section of the present document. The result from such a toxicity study in mice or some other animal can then be correlated to estimated maximum allowable concentrations for other animals, including humans, using techniques well known in the art. For example, the dosage conversion factor table of Freireich et al. [17] can be used. According to that conversion factor table, a conversion factor from mouse to man of about 1/12 is suggested, implying that if a maximum polymer concentration of X % is allowable in mice, the corresponding estimated maximum concentration in humans is about X/12%.

For example, toxicity studies in mouse have shown that a maximum polymer concentration of about 30% w/w can safely be injected in mouse without any side effects. This would then correspond to a concentration limit of about 2.5% w/w for human administration. Some of the above listed amphiphilic copolymers of the present invention, including PLURONIC®, have underwent clinical phase studies and extensive toxicity investigations.

The concentrations used for administration of the polymers can non-inventively be determined by the person skilled in the art based on the above-described procedures. It is expected that a polymer concentration of up to 30% w/w, such as up to 25, 20, 25 or 10% w/w, or up to 7.5% w/w, preferably 0.001 to 5 w/w %, more preferably at least 0.01% w/w, such as at least 0.1 w/w % can be suitable concentrations.

Suitable concentrations can be those that give a mean blood concentration below 5% w/w, probably less than 2.5% w/w and especially less than 1% w/w. A preferred concentration range is between 0.0001% w/w and 1% w/w polymer, such as more than 0.01% w/w, or more than 0.1% w/w.

The present invention can be used in connection with animal subjects, preferably mammal subjects and more preferably human subjects.

The active copolymers of present invention can be utilized for reducing the growth rate of tumors of different cancer lines and types. The present invention is applicable on several different types of cancers, including, but not limited to, human sarcomas and carcinomas, e.g. fibrosarcoma, myxosarcoma, liposarcoma, chondrosarcoma, osteogenic sarcoma, chordoma, angiosarcoma, enotheliosarcoma, lymphangiosarcoma, lymphangioendotheliosarcoma, synovioma, mesothelioma, Ewing's tumor, leiomyosarcoma, rhabdomyosarcoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, hepatoma, bile duct carcinoma, choriocarcinoma, seminoma, embryonal carcinoma, Wilms' tumor, cervical cancer, testicular tumor, lung cacinoma, small cell lung carcinoma, bladder carcinoma, epithelial carcinoma, glioma, astrocytoma, medulloblastoma, craniopharyngioma, ependymoma, hemangioblastoma, oligodendroglioma, melanoma, neuroblastoma and retinomblastoma, leukemias, e.g. acute lymphocytic leukemia (ALL), and acute myelocytic leukemia (myeloblastic, promyelocytic, myelomonocytic, monocytic and erytholeukemia), chronic leukemias (chronic myelocytic leukemia, chronic granulocytic leukemia and chronic lymphocytic leukemia), polycythemia vera, lymphoma (Hodgkin's disease and non-Hodgkin's disease), multiple myeloma, Waldenström's macroglobulinemia and heavy chain disease.

The pharmaceutical composition of the present invention can include one of the amphiphilic block copolymers of the invention. In an alternative embodiment, the composition comprises a mixture of at least two amphiphilic block copolymers of the invention.

Furthermore, the present invention can be used as a complement to other traditional cancer treatment techniques, e.g. irradiation, chemotherapy, hormone treatment, etc., to combat cancer in a patient.

The polymers of the invention may advantageously be used in connection with other chemotherapeutic drugs. In such a case, at least one such chemotherapeutic drug can be administered simultaneously with or sequentially relative administration of at least one amphiphilic copolymer of the present invention.

Examples of suitable chemotherapeutic agents that can be used in connection with the copolymers of the invention include:
alkylating agents, such as cisplatin, carboplatin, oxaplatin, mechloethamine, cyclophosphamide, chlorambucil:
anti-metabolites, such as methotrexate, azathioprine, mercaptopurine, thioguanine, fludarabine, pentostatin, cladribine, 5-fluorouracil, floxuridine, cytostine arabinoside;
anthracyclines, such as daunorubicin, doxorubicin, epirubicin, idarubicin, mitoxantrone;
vinca alkaloids, such as vincristine, vinblastine, vinorelbine, vindesine;
phodophyllotoxin, such as etoposide, teniposide;
taxanes, such as paclitaxel, docetaxel; and
topoisomerase inhibitors, such as irinotecan, topotecan, amsacrine, etoposide, etoposide phosphate, teniposide.

A possible theory for the growth inhibiting effect of the amphiphilic copolymers of the invention follows herein. The present invention is however not bound at all to this theory. The copolymers are preferably water soluble so that they, upon administration or in vitro, can reach and interact with the cancer cells. The hydrophobic part of the copolymers may then penetrate into the cell membrane and bind thereto. The hydrophilic parts prevent to copolymer from fully entering into the membrane. This means that the copolymers will typically be anchored in the cell surface. Once fixed in the membrane, the copolymers may exert their cell growth inhibiting action in different ways.

The amphiphilic copolymers can bind to growth factors and thereby inactivating them or at least prevent them from binding to and activating growth receptors in the cancer cell membranes. This has been seen in experiments with one of the amphiphilic block copolymers of the invention that is able to block the binding of Fibroblast Growth Factor 2 (FGF2, also denoted basic FGF) and Platelet Derived Growth Factor (PDGF) to the respective receptors on cell membranes.

In addition, the amphiphilic copolymer could block growth receptors in the membrane and prevent these receptors from pairing together, which are often necessary for forwarding a signal into the cell.

In addition, or alternatively, the amphiphilic copolymers can bind to growth receptors and thereby inactivating them or at least partly block them and thereby preventing growth factors from binding to and activating the receptors.

Some of the amphiphilic block copolymers of the present invention have previously been used in connection of anti-neoplastic agents. For example, it is known [18] that a combination of a selected PLURONIC® polymer and polyethylene oxide can be used to decrease the toxicity of an anti-neoplastic agent and for increasing the anti-cancer activity by i) increasing the stability of the agent in the blood stream, ii) making the agent more soluble or iii) improving the transport of the agent across cell membranes. It is also known [19] that PLURONIC® block copolymers affects several distinct drug resistance mechanisms including inhibition of drug efflux transporters, abolishing drug sequestration in acidic vesicles and inhibiting the gluthion/glutathione S-transferase detoxification system. All these mechanisms of drug resistance are energy-dependent and therefore ATP depletion induced by PLURONIC® block copolymers in multidrug-resistant cancer cells is considered as the reason for the chemosensitization experienced through the combined administration of anthracycline antibiotics and PLURONIC® copolymers.

However, it has not hitherto been realized that amphiphilic block copolymers, such as PLURONIC® copolymers, has anti-cancer effect per se in the form of inhibiting the proliferation and growth rate of cancer cells. Thus, it has not be realized that an effective anti-cancer medicament can comprise an amphiphilic block copolymer of the present invention without any of the prior art chemotherapeutical agents and still being effective in preventing or treating cancer.

A first aspect of the invention relates to a pharmaceutical composition comprising an amphiphilic block copolymer of the present invention as anti-cell proliferation or anti-cell-growth agent. This aspect also relates to the use of an amphiphilic block copolymer of the invention in the manufacture of an anti-cell-proliferation or anti-cell-growth medicament. The invention also encompassed an in vitro method of modulating, i.e. reducing or even inhibiting, the proliferation rate or growth rate of a cell, preferably a cancer cell. This method involves contacting the cell, preferably the cancer cell, with an amphiphilic block copolymer. The amphiphilic block copolymer is preferably added in the culture medium used for the cell. A further embodiment relates to an in vivo method of modulating, i.e. reducing, proliferation rate or cell growth rate of a cell, preferably a cancer cell. The method involves administering a pharmaceutical composition according the first aspect of the invention to a subject, where this subject is an animal subject, preferably a mammalian subject and more preferably a human subject.

A second aspect of the invention relates to a pharmaceutical composition comprising an amphiphilic block copolymer of the present invention as chemotherapeutic agent for treating or preventing cancer with the proviso that the amphiphilic block copolymer is not PLURONIC® F68 (average molecular weight 8 400 g/mol, average ethylene oxide content of 81.8±1.9%, a melt point of 52° C. and an average Brookfield viscosity of 1 000 cps at 77° C. and average chemical structure of HO—(CH₂CH₂O)₈₀—(CH(CH₃)CH₂O)₂₇—(CH₂CH₂O)₈₀—H). Another embodiment relates to the use of an amphiphilic block copolymer as chemotherapeutic agent (active anti-cancer agent) in the manufacture of a medicament for treating or preventing cancer with the proviso that the block copolymer is not PLURONIC® F68. This aspect also relates to a method of treating or preventing cancer in a subject, preferably a mammalian subject and more preferably a human subject. The method involves administering a pharmaceutical composition according to the second aspect to the subject.

A third aspect of the invention relates to a pharmaceutical composition comprising an amphiphilic block copolymer of the present invention for reducing or inhibiting a growth rate of cancer cells in a subject, preferably mammalian subject and more preferably a human subject, suffering from cancer. An embodiment of this aspect relates to the use of an amphiphilic block copolymer of the invention in the manufacture of a medicament for inhibiting or reducing the growth rate of cancer cells in the subject suffering from cancer. This aspect also relates to a method of reducing a growth rate of cancer cells in a subject suffering from cancer, where the method involves administering the pharmaceutical composition of the third aspect to the subject.

A fourth aspect of the invention relates to a pharmaceutical composition comprising an amphiphilic block copolymer represented by formula (IV):

HO—(CH₂CH₂O)ₙ—(CH(CH₃)CH₂O)ₘ—(CH₂CH₂O)ₚ—H (IV)

where m, n and p are each integer numbers, preferably multiple integers numbers for treating or preventing cancer in a subject, preferably mammalian subject and more preferably a human subject, with the proviso that the cancer is not colon cancer or rectal cancer. An embodiment teaches the use of an amphiphilic block copolymer represented by formula (IV) as a chemotherapeutic agent in the manufacture of a medicament for treating or preventing cancer in a subject with the proviso that said cancer is not colon or rectal cancer. This aspect also relates to a method of treating or preventing cancer different from colon cancer and rectal cancer in a subject by administering the pharmaceutical composition of the fourth aspect to the subject.

A fifth aspect of the invention relates a method, including an in vitro method, of blocking binding of a growth factor to a growth factor receptor on a cell membrane of a cell. The method comprises contacting the cell with an amphiphilic block copolymer according to the present invention.

### EXAMPLES

In the experiments different amphiphilic block copolymers are used. The PLURONIC® and TETRONIC® polymers were obtained from BASF Corporation. The amphiphilic block copolymer denoted DORVAL 1 is a variant of a PLURONIC® polymer but with the central propylene oxide chain exchanged for a corresponding polystyrene chain. That block copolymer was ordered from Polymer Source Inc., Canada. The copolymer has the following schematic structure: (EO)ₓ-(St)_{y}-(EO)ₓ, where the x≈70 and y≈27, Mₙ: PEO(3100)-PSt(2800)-PEO(3100) and M_{w}/Mₙ=1.11.

### Growth Rate Inhibition of Breast Cancer Cells

The effect of PLURONIC® F127 was tested on cell growth of human breast cancer cell lines cultured in vitro. The growth rate was measured with incorporation of ³H-thymidine. An aggressively growing breast cancer cell line, MCF-7, and a more slowly growing breast cancer cell line, SK-BR-3, were examined.

Briefly, about 3×10⁴ cells were seeded in 24-well microtiter plates in 1.0 ml RPMI (Roswell Park Memorial Institute) culture medium supplemented with 10% FCS (Fetal Calf Serum), insulin (1 mg/100 ml) and 1% antibiotic and incubated (humidified air, 5% CO₂, 37° C.) over night. After overnight incubation, the medium was removed by vacuum suction using sterilized Pasteur pipettes and was exchanged by 1.0 ml RPMI with 0.1% FCS per well. Following overnight incubation, RPMI supplemented with different concentrations (0.01, 0.1, 0.3, 1 and 2% by weight) of the growth rate modulating PLURONIC® F127 and/or 10% FCS were added to cells (n=3). In all wells except control wells and wells with 1% F127, FCS was also added to the wells. The wells were incubated about 15 hours.

1 μCi/ml ³H-thymidine (Amersham-Pharmacia Biotech) was added per well and incubated in 4 hours. At the end of the labeling period, the medium was removed and the cells were rinsed twice in PBS and fixed with chilled (4° C.) 10% TCA (trichloroacetic acid) for 10 minutes. TCA was then removed and the monolayers were washed in 95% ethanol and air-dried at room temperature for 20 minutes.

Thereafter, 1.0 ml 0.2 M NaOH was added per well and incubated at least one hour in room temperature for dissolving the cells. 1.0 ml of each well-content was diluted in 4 ml of Highsafe II scintillation solution in 5 ml scintillation tubes. Radioactivity was measured on a beta counter.

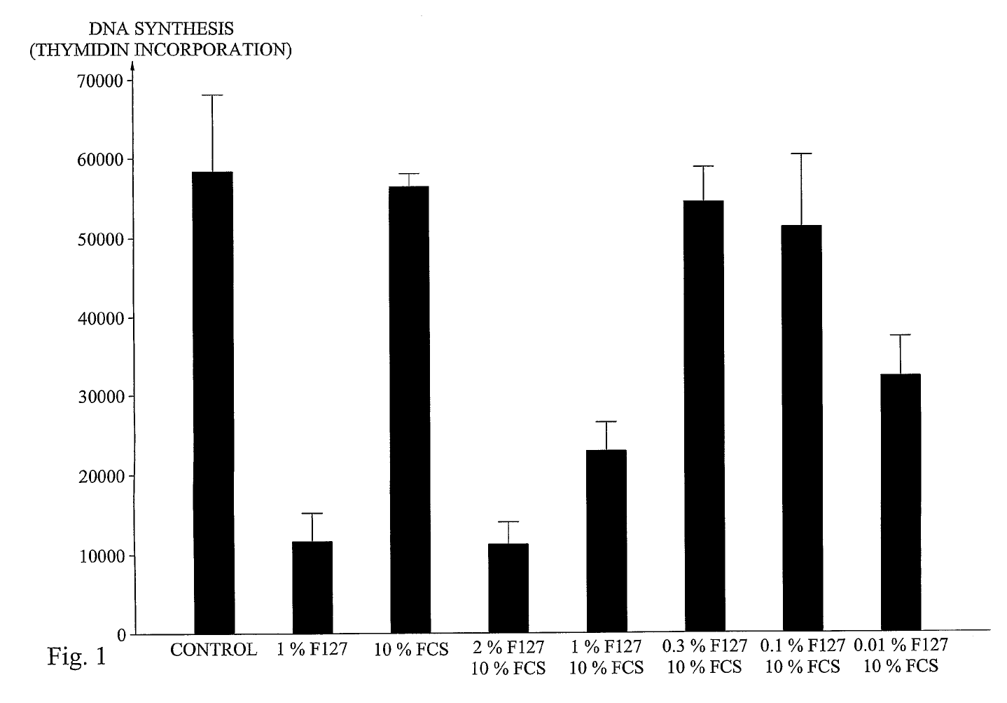

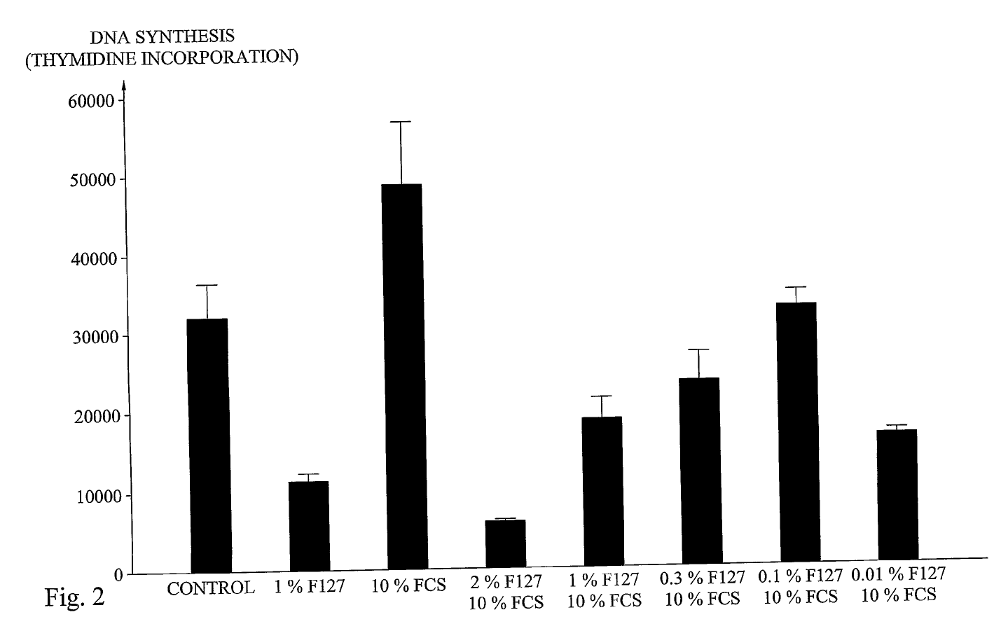

As can be seen in the FIGS. 1 and 2, PLURONIC® F127 markedly reduced growth of both breast cancer cell lines. The effect was most pronounced in the rapidly growing cancer (MCF-7) were 1% PLURONIC® F127 reduced cell proliferation by approximately 80%. The effect was also pronounced in SK-BR-3 were the growth was reduced by approximately 60%.

Addition of a growth stimulus FCS increased proliferation in SK-BR-3 but not in MCF-7, probably because MCF-7 by itself proliferates at maximum rate. In SK-BR-3 the effect of PLURONIC® F127 was increased in FCS-stimulated cells compared to unstimulated cells.

PLURONIC® F127 was effective as an inhibitor of proliferation even at the lowest concentration tested (0.01% w/w).

### DNA Synthesis Inhibition on Stimulated Smooth Muscle Cells

The above-described growth rate experiments were also conducted in vitro on vascular smooth muscle cells from man, rat and rabbit. The experiments confirmed that PLURONIC® F127 inhibited DNA-syntheses in a dose-dependent manner as measured by incorporation of thymidine in growth-stimulated (presence of 10% FCS) vascular smooth muscle cells from man, rat and rabbit. However, PLURONIC® F127 did not affect the growth rate of unstimulated muscle cells.

Briefly, for large vessels, the dissected vessel was cut open and the endothelial was scraped off by a scalpel. The vessel was turned and further adventitia was scraped off. For smaller vessels, the endothelial was removed by flushing the vessel lumen with 0.1% Triton X-100 for 10 s, followed by flushing with DMEM (Dulbecco's Modified Eagle's Medium) culture medium.

The vessel was hacked into smaller pieces, about 1×1 mm. The vessel pieces were transferred to cell culture bottle with DMEM supplemented with 10% FCS and 1% antibiotic for incubation in 10 days. For human cells, the DMEM medium was also supplemented with 10% human serum (NHS).

In a passage of the cells, the spent culture medium was pipetted off and discarded. The cells were rinsed twice by addition of PBS (10 ml/75 cm² flask) to the flasks, while being careful not to disturb the cell monolayer. The monolayer is rinsed by gently rocking the flask back and forth. The PBS was removed and discarded. Trypsin (3.5 ml/75 cm² flask) was added to the flasks and the flasks were rocked gently to ensure that the entire monolayers were covered with the trypsin solution.

The flasks were incubated about 3-5 minutes until the cells began to detach. 3.5 ml 10% FCS was added per flask to “neutralize” the trypsin and the solutions were pipetted up and down until the cells were dispersed into a single cell suspension.

The solution was centrifuged at 300 g for 5 minutes and the supernatant was removed and discarded. The cell pellet was solved in DMEM and transferred to two new culture flasks.

The cell growth rate (DNA synthesis) experiments were then conducted in the same way as for the two breast cancer cell lines described above.

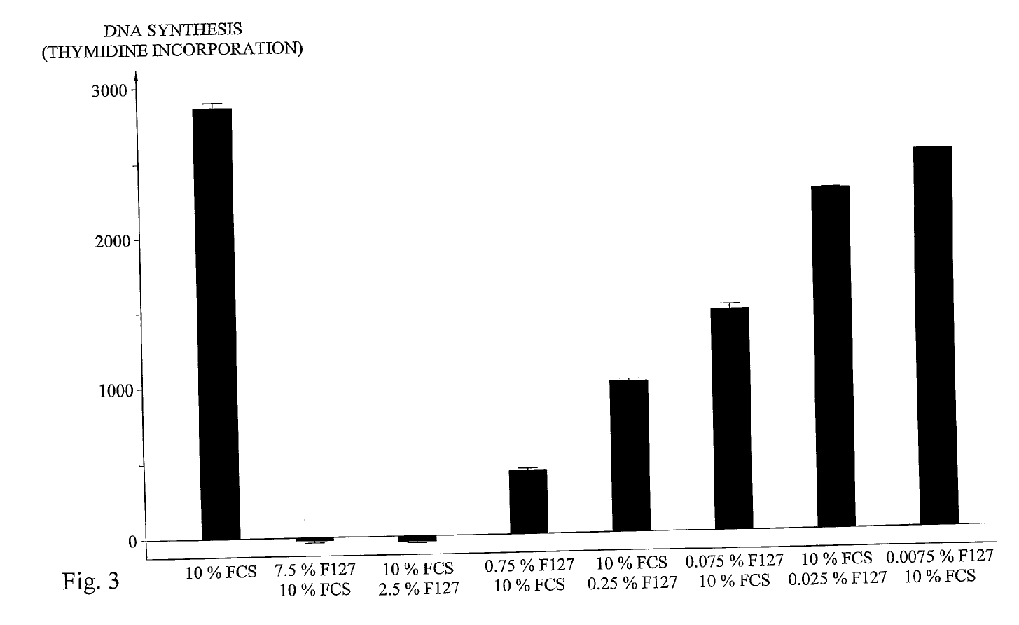

FIG. 3 illustrates the results on growth rate modulation of PLURONIC® F127 on human vascular smooth muscle cells. It is seen that PLURONIC® F127 has a dose-dependent proliferation rate inhibition on the FCS-stimulated muscle cells. Comparative results were also obtained from rat and rabbit vascular smooth muscle cells.

In a comparative study, the DNA synthesis inhibiting effect on FCS-stimulated (10%) smooth muscle cells of F127 and other PLURONIC® polymers were investigated. The results are presented in Table V below normalized to the DNA synthesis (as determined using the above described thymidine-based method) of control cells grown in medium supplemented with 10% FCS. The tested cells were grown in medium supplemented with 10% FCS and a copolymer at a concentration of 10, 1 or 0.1 mg/ml. In these tests the DNA synthesis of the control cells is set to 100% and the tested substances are expressed as percentage of the DNS synthesis of the control cells.

TABLE V
DNA synthesis inhibition

| | Normalized DNA synthesis | | |
|---|---|---|---|
| PLURONIC ® | relative 10% FCS cells (%) | | |
| polymer | 10 mg/ml | 1 mg/ml | 0.1 mg/ml |
| L31 | −39.0 ± 6.8 | 3.4 ± 6.8 | 91.5 ± 4.1 |
| F38 | 102.7 ± 11.6 | 68.5 ± 4.1 | 83.6 ± 8.9 |
| F68 | 131.8 ± 6.8 | 93.2 ± 6.8 | 97.7 ± 5.7 |
| F98 | 49.3 ± 6.9 | 61.6 ± 1.4 | 52.1 ± 8.2 |
| L121 | 47.7 ± 2.3 | 95.5 ± 0.9 | 127.3 ± 11.4 |
| P123 | −9.6 ± 1.6 | 68.0 ± 8.0 | 108.8 ± 5.6 |
| F127 | 1.4 ± 3.4 | 30.1 ± 4.1 | 56.2 ± 7.5 |

These experiments confirm that PLURONIC® polymers can be used for inhibiting DNA synthesis. The experiments also show that PLURONIC® F68 does not seem to have any such inhibiting effect. At the highest tested concentration (10 mg/ml), L31 had a tendency of killing the cells.

### Cell Growth Inhibition on Stimulated Smooth Muscle Cells

In order to confirm that the inhibition of DNA synthesis was due to reduced cell numbers, i.e. proliferation rate inhibition, a calorimetric method was used to measure cell numbers following PLURONIC® F127 treatment.

Vascular smooth muscle cells from rat aorta were obtained using the above-described procedure. 5 000 rat aorta cells in 200 μl DMEM supplemented by 10% FCS were seeded per well in a CellTiter 96™ Aqueous plate (Promega). The cells were allowed to incubate for about 1 day. The medium was pipetted off and discarded and exchanged by 200 μl DMEM with 0.1% FCS per well.

After 2 days, cell DMEM medium (negative control), DMEM medium with 10% FCS (positive control), DMEM medium with 1 or 5% PLURONIC® F127 or DMEM medium with 10% FCS and 1 or 5% PLURONIC® F127 was added to different wells (n=3) and incubated according to the protocol of the CellTiter 96™ Non-Radioactive Cell Proliferation/Cytoxicty Assay manufacturer.

The wells were then washed three times with PBS according to the manufactures protocol and 20 μl MTS solution was then added per well. The plate was incubated in 1-4 hours and the absorbance was measured at 490 nm.

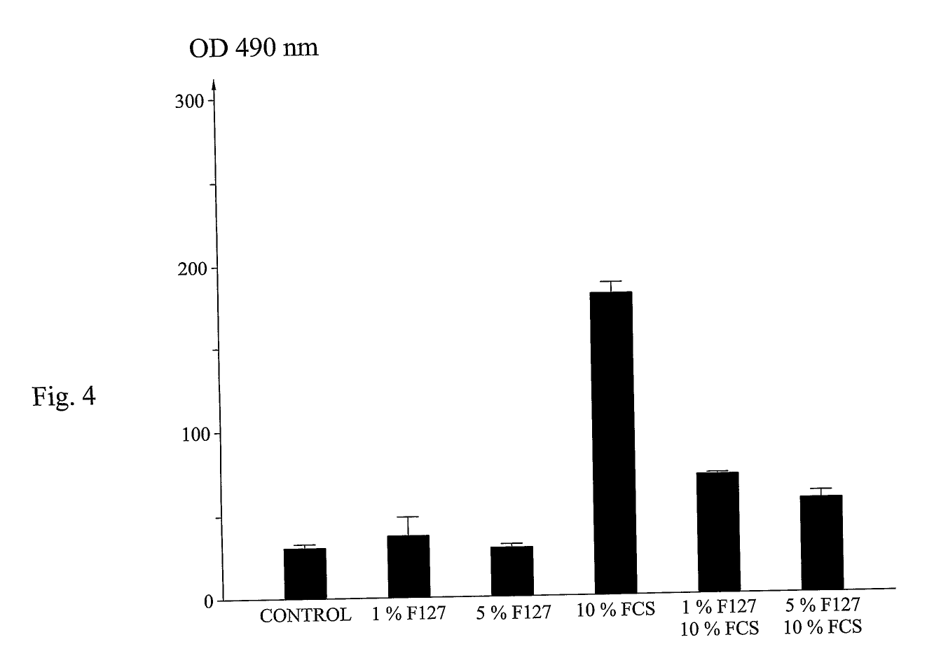

The results are illustrated in FIG. 4. It is seen in the diagram that the colorimetric method confirms the cell proliferation rate inhibition of PLURONIC® F127 observed using the above-described DNA synthesis method. PLURONIC® F127 inhibited the growth stimulating effect of FCS but did not have any effect on unstimulated cells.

### Cell Mediated Cytoxicity

In order to determine whether the cell proliferation inhibiting effect of PLURONIC® F127 is due to any toxic effect of the copolymer on the cells, a cell mediated cytotoxicity test was performed where the cytotoxicity of PLURONIC® F127 was compared to 1% Triton X-100, PEG 10 000 and another PLURONIC® polymer P123.

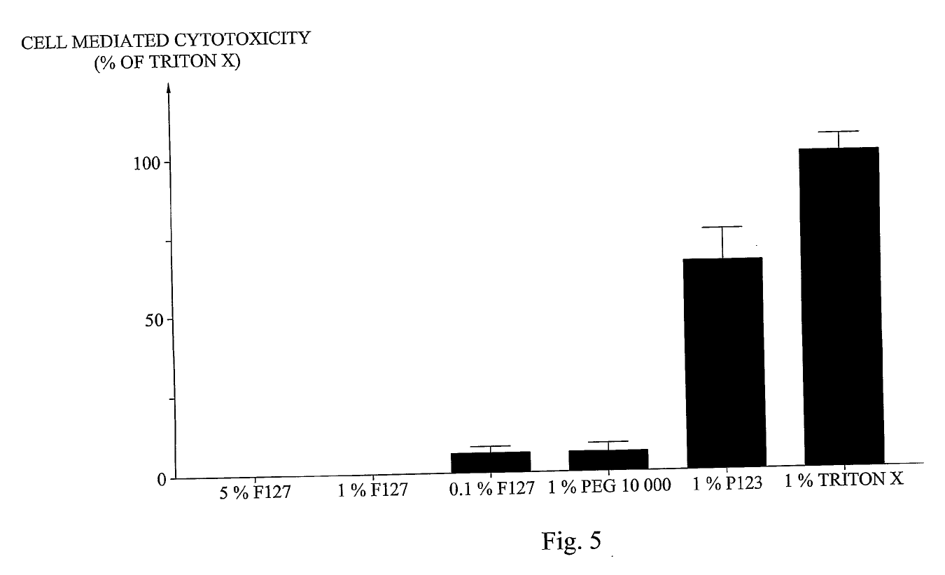

The above-described procedure using the CellTiter 96™ Non-Radioactive Cell Proliferation/Cytotoxicity Assay from Promega was performed using different concentrations of PLURONIC® F127, 1% Triton X-100, 1% PEG 10 000 and 1% PLURONIC® P123. FIG. 5 illustrates the cytotoxic effect of different PLURONIC® F127 concentrations expressed in percentage of the cytotoxicity of Triton X-100. PLURONIC® F127 exhibit no cell toxicity even at the highest tested concentration of 5%. However, the other tested PLURONIC® polymer P123 exhibited comparatively significant higher cytotoxicity.

### Comparative Study of Amphiphilic Block Copolymers

The growth rate inhibiting effect of other amphiphilic block copolymers besides PLURONIC® F127, including PLURONIC® F38, F68, F87, F98, P105, F108, and TETRONIC® T908, T1307 from BASF corporation and DORVAL 1 from Polymer Source Inc., were tested on stimulated (10% FCS) human vascular smooth muscle cells.

The experiments were performed in the same manner as the thymidine-based DNA synthesis experiment described above and illustrated in FIG. 3, with the difference that three concentrations 1, 0.1 and 0.01% w/w were tested per block copolymer.

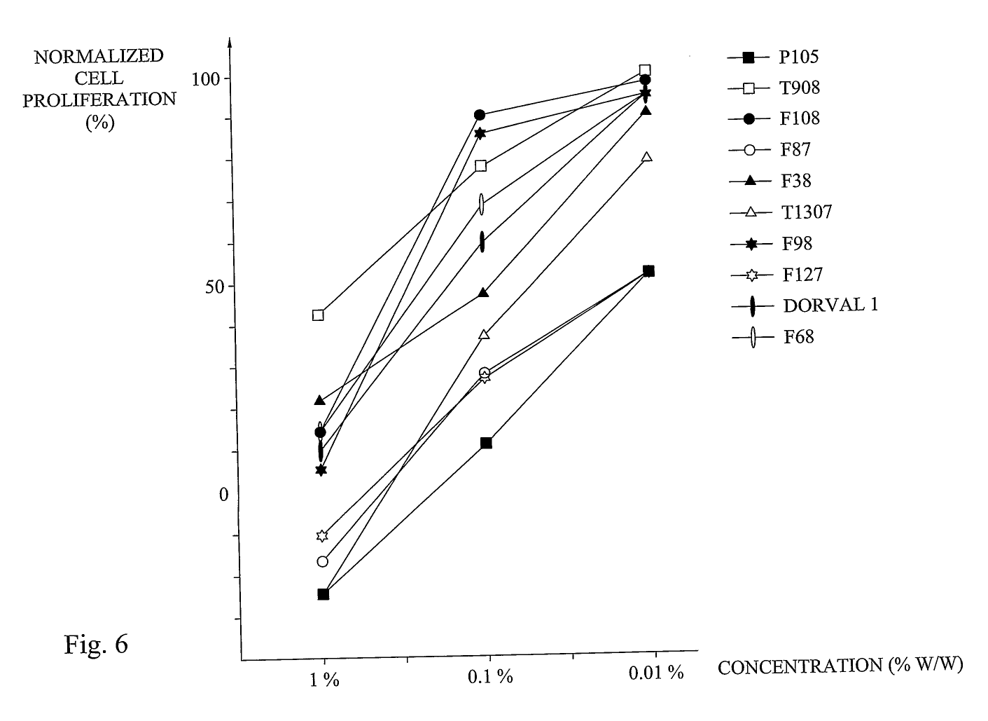

The results of the growth rate inhibition are presented in FIG. 6, where the growth rates have been normalized relative the highest measured cell growth rate (T908 and 0.01% w/w).

It can be seen in the figure that the copolymer having the highest hydrophilic content (about 80%), i.e. F38, F68, F108 and T908, showed the lowest cell growth inhibiting effect on the FCS-stimulated smooth muscle cells. The copolymer F87 had similar effect as F127, while P105 achieved the highest inhibitory effect under the present experimental settings.

### Binding Experiments

Test experiments were conducted to determine whether the growth rate inhibitory effect of PLURONIC® F127 might be mediated through the blocking of the binding of different growth factors to respective receptors on rat aorta smooth muscle cells.

Rat aorta cells prepared as previously described added in culture medium (+10% FCs) to wells of a 24-well microtiter plate at a concentration of about 5 000 cells per well. The plate was incubated over night to allow the cells to form a layer of the well bottoms. The culture medium was then replaced with culture medium supplemented with 0.1% FCS and allowed to incubate for two days.

The culture medium was then removed and the wells were washed twice with PBS. 150 μl NaCl solution with different concentrations of PLURONIC® F127 (2, 1, 0.1, 0.01, 0.001 and 0.0001% w/w) was added together with 1 μl ¹²⁵I-FGF2 (Radioactively labeled Fibroblast Growth Factor 2) or 1 μl ¹²⁵I-PDGF (Platelet Derived Growth Factor) diluted in a buffer solution (0.237 M NaCl, 0.0054 M KCl, 0.00044 M KH₂PO₄, 0.00126 M CaCl₂, 0.00018 M MgSO₄, 0.020 M HEPES and 0.3% BSA) and incubated in 30 minutes at 37° C. The wells were then washed five times with PBS (Phosphate Buffered Saline; 0.2 g KCl, 0.2 g KH₂PO₄, 1.35 g Na₂HPO₄ and 8.0 g NaCl per 1 000 ml distillated H₂O). 1 ml 0.2 M NaOH was then added per well and the plate was placed in a refrigerator over night. The amount of binding of the radioactively labeled growth factors was then determined through traditional gamma measurement.

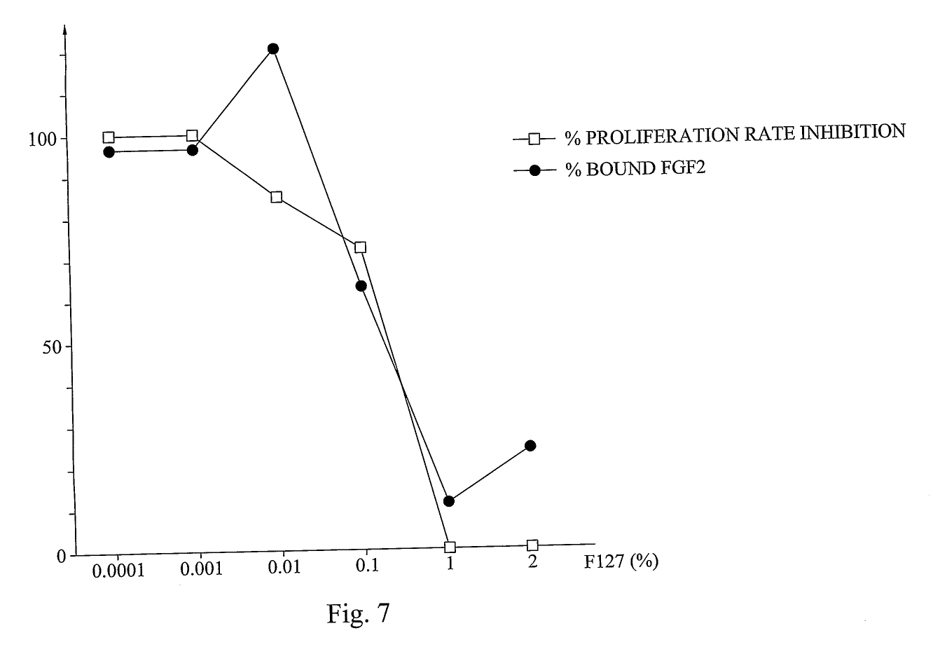

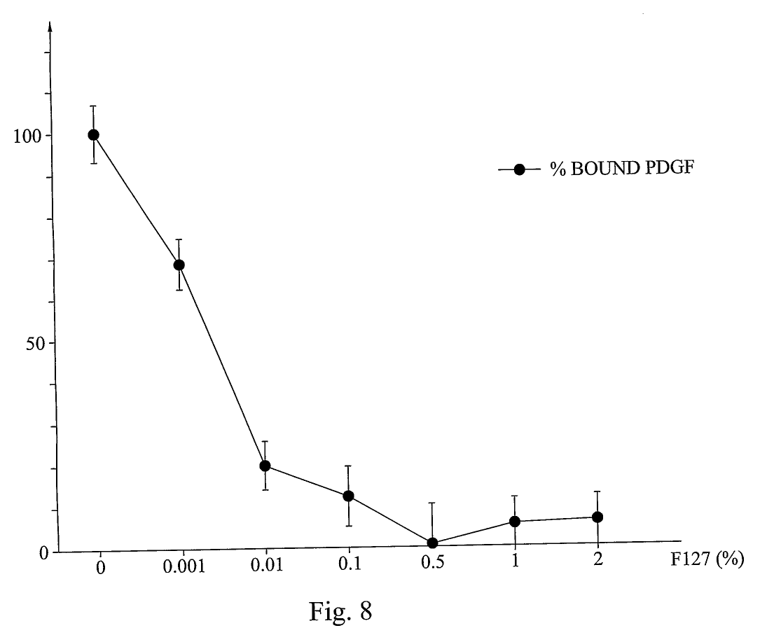

It was concluded that PLURONIC® blocks the binding of the two growth factors to their respective receptors on the cells in a dose-dependent manner. In addition, as is illustrated in FIG. 7, there is a very high correlation between the F127 concentrations needed for the growth inhibitory effects and for the inhibition of FGF2 binding. FIG. 8 illustrates the corresponding binding blocking effect of the F127 polymer on radioactively labeled PDGF. As a consequence, the blocking of this growth factor binding to receptors in the cell membrane can be at least one of the mechanisms of the growth rate inhibition of the amphiphilic block copolymers of the present invention.

### Toxicity Study in Mouse

Experiments were conducted to investigate whether PLURONIC® P105 produces toxic reactions after intravenous administration in mice.

Ten NMRI albino mice, weighing about 25 g at arrival, were used for the experiment. The animals were obtained from Scanbur B K, and were conditioned for one week before start of the study. The animals were provided with food and water ad libitum.

The active substance PLURONIC® P105 was provided in two bulk solutions of 10 and 50% by weight, respectively, of the copolymer in NaCl (9 mg/l) for 10% solution and in NaCl (9 mg/l) and PEG for the 50% solution.

The animals were separated in five groups and were treated i.v. in a tail vein, once daily for 5 days. The injected volume for all groups was 150 μl. The injections were performed during 10 seconds.
Group 1: 50% PLURONIC® P105 (n=2)
Group 2: 40% PLURONIC® P105 (n=2)
Group 3: 30% PLURONIC® P105 (n=2)
Group 4: 20% PLURONIC® P105 (n=2)
Group 5: 10% PLURONIC® P105 (n=2)

Body weights were to be recorded before the first administration and at day 6. The animals were observed for clinical signs of toxicity (fur quality, salivation, lacrimation, diarrhea, respiration, motor disturbances, apathy, tremor, convulsions and coma) during 0-30 minutes and at 1, 2, 3, 4, 8, 24, 48 and 72 hours after administration of the test substance.

An exploratory study was performed on 2 mice treated with 10% PLURONIC® P105 and 2 mice treated with 50% PLURONIC® P105. The animals treated with the lowest P105 concentration were found to tolerate the repeated treatment well but those treated with 50% P105 showed edematous and haemolytic tails already at the second injection. In addition, these two animals showed decreased motor behavior and were subsequently euthanized at the third day after start of treatment. At this point it was decided to treat 6 mice with the 50% formulation diluted with saline to 40%, 30% and 20%.

Animals treated with 40% P105 showed slight haemolytic discoloration of the tails at day 2 which persisted during the treatment period. Some edema was noted. These animals also showed a decreased weight gain, see Table VI.

Animals injected with the 20% and 30% dilutions were found to tolerate the treatment well.

TABLE VI
weight gain of mice

| PLURONIC ® P105 concentration | Average weight gain at day 6 (g) |
|---|---|
| 40% | 0.1 |
| 30% | 7.2 |
| 20% | 14.9 |
| 10% | 6.1 |

### Hollow-Fiber Implantation Test

Tests were conducted to investigate whether PLURONIC® P105 inhibited tumor cell growth in a hollow-fiber model in mouse.

Eighteen NMRI albino male mice, weighing about 25 g at arrival, were used for the experiment. The animals were obtained from Scanbur B K, and were conditioned for one week before start of the study. The animals were provided with food and water ad libitum.

The filling of the fibers was performed at Uppsala University Hospital, Department of Clinical Pharmacology. The fibers were loaded with the following tumor cells: yellow fibers with U936/gtb and blue fibers with H69.

After shaving and disinfection a small skin incision was, under isofluran anesthesia, made on the back of the animals. Three fibers, two yellow and one blue, were implanted subcutaneously in a randomized manner and the skin incision was closed using staples.

The animals were separated into three groups and were treated as follows intravenously in a tail vein once daily for 5 days starting immediately after implantation:
Group 1: 10% PLURONIC® P105 diluted in NaCl (n=6)
Group 2: 25% PLURONIC® P105 diluted in PEG and NaCl (n=6)
Group 3: vehicle (n=6)

Body weights were recorded before the first administration and before euthanasia. The animals were checked daily for signs of change in food intake, activity, etc. as signs of a change in general health status. Six days after fiber implantations the animals were anaesthetized with isofluran and approximately 250 μl blood was obtained from the orbital plexus for haematology. Thereafter the animals were euthanized by cervical dislocation and the fibers were explanted and placed in cell culture media (37° C.) before evaluation of cell density and viability.

Statistical evaluation was performed with use of Graph Pad Prism version 4 (Graph Pad Software Inc., San Diego, U.S.) on a HP Compac dx 2000 computer under Windows XP. One-way ANOVA with the Tukey's multiple comparisons test was used to test statistically differences in haematology parameters between the groups of treatment. Paired t-test was used to test statistically differences in weights before and after treatments.

There were no overt signs of change in health status in any of the animals. There were no statistically significant differences within groups regarding weights before and after treatment.

Statistically significant differences between groups were found in the haematology parameters RBs (p=0.0127 group 1 vs. group 3 and group 2 vs. group 3), HGB (p=0.021, group 1 vs. group 3 and group 2 vs. group 3) and PLT (p=0.0006, group 1 vs. group 3 and group 2 vs. group 3), see Table VII.

TABLE VII
haematology parameters

| Group | WBC (10⁹/l) | RBC (10¹²/l) | HGB (g/l) | HCT (l/l) | PLT 10⁹/l |
|---|---|---|---|---|---|
| 1 | 9.6 ± 1.1 | 7.87 ± 0.56 | 132.8 ± 8.9 | 0.460 ± 0.144 | 1014.0 ± 103.5 |
| 2 | 7.6 ± 1.6 | 7.94 ± 0.47 | 134.7 ± 6.1 | 0.403 ± 0.021 | 947.0 ± 61.6 |
| 3 | 8.9 ± 1.6 | 8.65 ± 0.15 | 148.2 ± 3.8 | 0.439 ± 0.015 | 744.7 ± 107.0 |

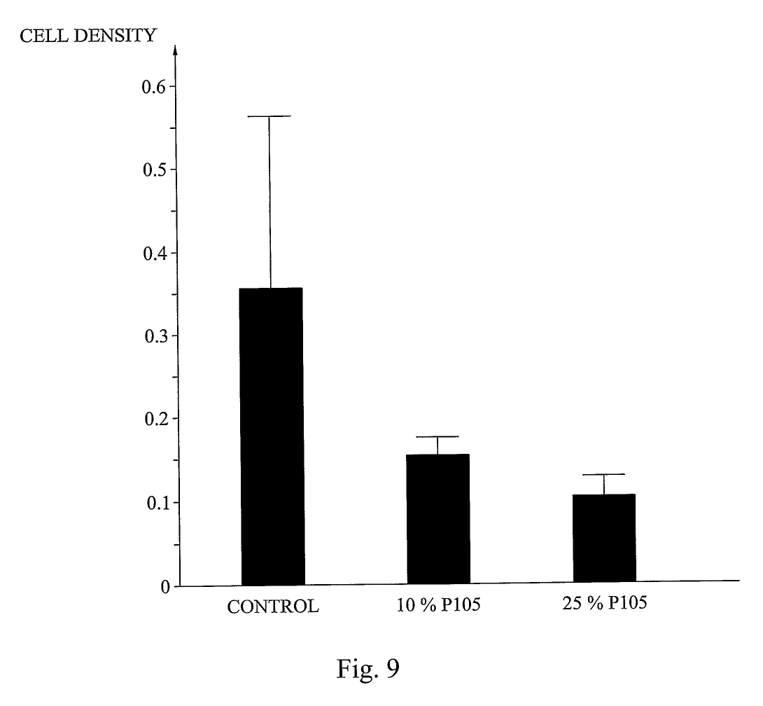

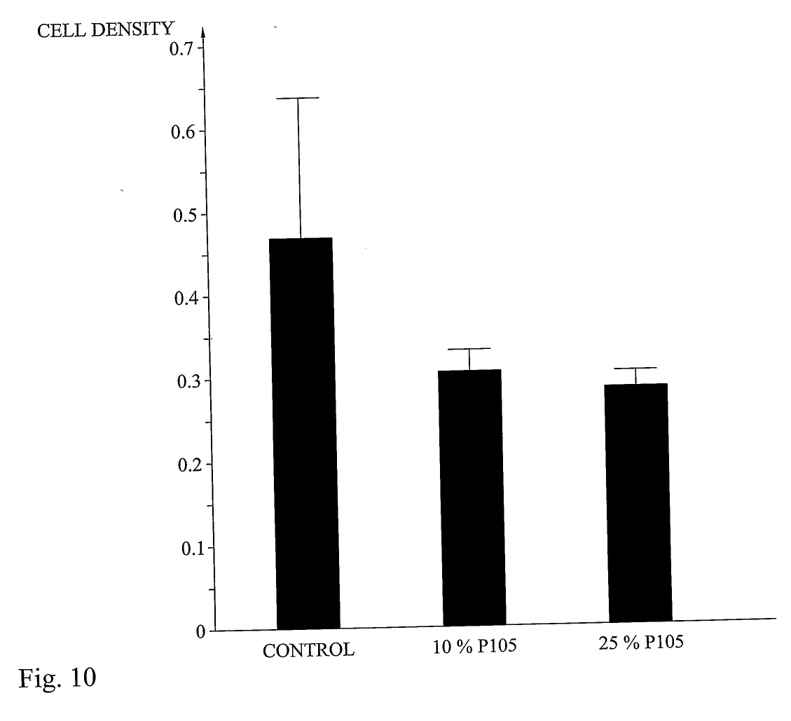

Cell density in fibers was significantly reduced (p<0.05) in U937/gtb containing fibers treated with the high dose of PLURONIC® P105 compared to control. A similar trend was observed also for the cells implanted in animals treated with the low dose of P105, see FIG. 9. A tendency towards lower mean cell density values was also observed for the H69 cell line in animals treated with the copolymer, see FIG. 10.

### In Vitro Estimation of the Cytotoxic Activity of Co-Polymers

The current study aims at investigating the cytotoxic activity of different PLURONIC® and TETRONIC® copolymers. As model systems, a well defined panel of 10 selected human tumor cell lines and one additional prostate cancer cell lines are used. Three compounds, selected after screening in the sensitive lymphoma cell line U937/gtb, are investigated in all cell lines.

A model system used in this study is a cell line panel of ten human tumor cell lines [3]. This concept originates from the National Cancer Institute (NCI) in the U.S., where a cell line panel with approximately 60 different cell lines (representing most forms of human cancer) is commonly used to define the activity profile of a new compound [4]. The cell line panel can successfully classify agents as being related to a specific mechanistic group (e.g. antimetabolites, alkylators, topoisomerase II inhibitors) by the use of correlation analysis [5]. It has earlier been demonstrated that a more limited number (10) of human tumor cell lines representing defined kinds of cytotoxic drug resistance can successfully be used for the initial evaluation and preliminary mechanistic classification of anticancer agents [6].

Tumor cells can gain resistance to cytotoxic drugs, and examples of known resistance mechanisms are the P-glycoprotein (Pgp) and multidrug resistance associated protein (MRP), increased activity of cellular detoxification systems, altered function of nuclear target enzymes like topoisomerase II (topo II) as well as altered tubulin binding/function and subcellular redistribution of the drug. The cell line panel used contains cell lines expressing some of these phenotypes [3].

The drug efflux pumps, e g Pgp and MRP, display low specificity for substrates and thus contribute to decreased sensitivity to agents of various classes, e g vinca alkaloids, anthracyclins, taxanes, epipodophyllotoxines and other drugs [7].

Primary cultures of human tumor cells are an alternative model system that has received relatively little attention in the context of new drug screening and development. However, it has been demonstrated that in vitro assays performed on primary cultures from different tumors correlates well with clinical tumor-type specific activity [8].

Combining different cytotoxic drugs and creating drug preparations including compounds that increase drug uptake or drug effect is a growing field in cancer chemotherapy. Numerous methods of performing and interpreting preclinical studies on drug interactions have been proposed. When data from single agents and their combinations are available at fixed concentrations, the “multiplicative concept” (additive model) is commonly used. Here, an additive interaction is defined as a combination of two drugs which results in a surviving fraction that equals the product of the surviving fractions of the single agents, which would indicate an independent action of the drugs [9]. If the effect of the combination surmounts the additive effect, the interaction is synergistic.

To evaluate the activity patterns of the drugs a human cell line panel of four sensitive parental cell lines, five drug resistant sublines, representing different mechanisms of resistance, and one cell line with primary resistance was used. The cell lines included were the myeloma cell line RPMI 8226/S and its sublines 8226/Dox40 and 8226/LR-5 (kind gifts from W. S. Dalton, Dept of Medicine, Arizona Cancer Center, University of Arizona, Tucson, Ariz.), the lymphoma cell lines U-937/gtb and U-937-Vcr (kind gifts from K. Nilsson, Dept of Pathology, University of Uppsala, Sweden), the SCLC cell line NCI-H69 and its subline H69AR (American Type Culture Collection; ATCC, Rockville, Md.), the renal adenocarcinoma cell line ACHN (ATCC) and the leukaemic cell line CCRF-CEM and its subline CEM/VM-1 (kind gifts from W. T. Beck, Dept of Pharmacology, College of Medicine, University of Tennessee, Memphis, Tenn.).

The 8226/Dox40 was selected for doxorubicin resistance and shows the classical MDR phenotype with overexpression of P-glycoprotein 170 (Pgp; [10]. The 8226/LR-5 was selected for melphalan resistance, proposed to be associated with increased levels of GSH [11]. The U-937-Vcr was selected for vincristine resistance, proposed to be tubulin associated [12]. The H69AR, selected for doxorubicin resistance, expresses a MDR phenotype proposed to be mediated by MRP [13]. The CEM/VM-1, selected for teniposide resistance, expresses an atypical MDR, which is proposed to be topoisomerase II (topoII) associated [14]. The exact mechanism of resistance for the primary resistant ACHN cell line is not known and may be multifactorial [15].

The cell lines were grown in complete culture medium described below at 37° C. in humidified atmosphere containing 5% CO₂. The 8226/Dox40 was treated once a month with doxorubicin at 0.24 μg/ml and the 8226/LR-5 at each change of medium with melphalan at 1.53 μg/ml. The U-937-Vcr was continuously cultured in presence of 10 ng/ml of vincristine and the H69AR was alternately fed with drug free medium and medium containing 0.46 μg/ml of doxorubicin. The CEM/VM-1 cell line was cultured in drug free medium without any loss of resistance for a period of 6-8 months. The resistance patterns of the cell lines were routinely confirmed in control experiments.

Human prostate cancer PC-3 cells were obtained from the American Type Culture Collection (Rockville, Md.). They were grown in Dulbecco's modified Eagle medium supplemented with 10% fetal bovine serum, penicillin G and streptomycin.

TABLE VIII
Human tumor cell lines

| | | Selecting | Associated |
|---|---|---|---|
| Cell line | Origin | agent | resistance |
| CCRF-CEM | Leukemia | | |
| CEM/VM-1 | Leukemia | teniposide | topoisomerase II |
| ACHN | Renal cancer | | primary resistance |
| NCI-H69 | Small cell lung cancer | | |
| H69AR | Small cell lung cancer | doxorubicin | MRP |
| RPMI 8226/S | Myeloma | | |
| 8226/dox40 | Myeloma | doxorubicin | Pgp |
| 8226/LR5 | Myeloma | melphalan | glutathione |
| U-937 GTP | Lymphoma | | |
| U-937-vcr | Lymphoma | vincristin | tubulin |
| PC-3 | Prostate cancer | | |

A complete medium consisting of carbonate buffered culture medium RPMI-1640 (HyClone, Cramlington, UK) supplemented with 10% inactivated FCS, 2 mM glutamine, 50 μg/ml of streptomycin and 60 μg/ml of penicillin was used throughout for cell lines. FDA (Sigma, St Louis, Mo.) was dissolved in DMSO and kept frozen (−20° C.) as a stock solution protected from light.

The test compounds were dissolved according to the Table IX below.

TABLE IX
Test substances

| | | | NaCl | |
|---|---|---|---|---|
| Copolymer | Amount agent (g) | PEG (g) | (9 mg/l) (g) | 95% EtOH (g) |
| L31 | 0.6 | 1.0 | | 0.2 |
| L121 | 0.6 | 1.0 | | 0.2 |
| F38 | 0.6 | | 1.0 | 0.2 |
| F127 | 5 | | 4 | 1 |
| F68 | 5 | | 4 | 1 |
| T1307 | 0.6 | 1.0 | | 0.2 |
| P105 | 0.6 | | 1.2 | |
| P84 | 0.6 | 1.0 | | 0.2 |
| F87 | 0.6 | | 1.0 | 0.2 |
| P123 | 0.6 | 1.0 | | 0.2 |
| F88 | 0.6 | 1.0 | | 0.2 |

The major part of primary screening was made on a first batch of substances (F127 and F68) dissolved in sodium chloride and ethanol only. In several experiments the activity was compared between copolymers dissolved in NaCl and ethanol and dissolved in PEG and ethanol, and there were no significant differences in potency. For simplicity the concentration of test substance in all received vials is assumed to be 50% w/w. Further dilutions from these were made with phosphate buffered saline (PBS; Sigma Aldrich) to clear solutions.

Tumor cells were seeded in the drug prepared 96-well plates at a cell density of about 20 000 cells/well.

A fluorometric microculture cytotoxicity assay (FMCA) based on measurement of fluorescence generated from hydrolysis of FDA to fluorescein by cells with intact plasma membranes and as previously described in detail [16] were used. The plates were incubated at 37° C. in humidified atmosphere containing 5% CO₂ for 72 hours. At the end of the incubation period the plates were centrifuged (1000 rpm, 5 minutes) and the medium was removed by aspiration. After one wash in PBS, 100 μl/well of FDA dissolved in a physiological buffer (10 μg/ml) was added. The plates were incubated for 45 minutes and the generated fluorescence from each well was measured in a 96-well scanning fluorometer (Fluoroscan II, Labsystems Oy, Helsinki, Finland). The fluorescence is proportional to the number of intact cells in the well.

Quality criteria for a successful analysis included a fluorescence signal in the control wells of more than five times mean blank value, a mean coefficient of variation (CV) in the control wells of less than 30% and more than 70% tumor cells in the cell preparation prior to incubation. Experiments were performed twice, mean values are used throughout.

Cell survival is presented as survival index (SI), defined as the fluorescence in the experimental wells in percent of that in the control wells, with values in the blank wells subtracted. All cell line experiments were performed at least two times, and all data was included in the analysis.

Data from the cell line panel experiments was compared with a database containing data from more than 150 different compounds including the most commonly used cytotoxic agents. For this purpose, an IC50 was calculated for every drug and cell line, defined as the drug concentration inducing a survival index of 50% using simple log-linear regression. The set of ten IC50 values for each drug was correlated using Pearson's correlation coefficient with the corresponding data set from all other drugs in the database. From these IC50s an activity pattern was also displayed using Delta, defined as the deviation of the log IC50 of one cell line from the mean log IC50 in the cell line panel. These calculations were performed according to Dhar et al [3], modified from the procedures used at the National Cancer Institute (www.dtp.nci.nih.gov).

Concentration-effect data from both the cell line panel were fitted to a sigmoidal dose-response equation with variable slope, using non-linear regression in the GraphPad Prism software (GraphPad Software, San Diego, Calif.). 0 and 100% cell survival was set as maximum effect and baseline, respectively, and the EC50 (concentration giving 50% effect) was predicted by the curve fitting. Resistance factors were calculated as the ratio between the EC50 in the resistant and parental cell line in the cell line pairs [3].

The compounds retained their cytotoxic activity after 4 weeks storage in microtiterplates at −70° C. The concentration-effect curves were similar when using plates that had been stored for 4 weeks and when using freshly prepared plates (not shown).

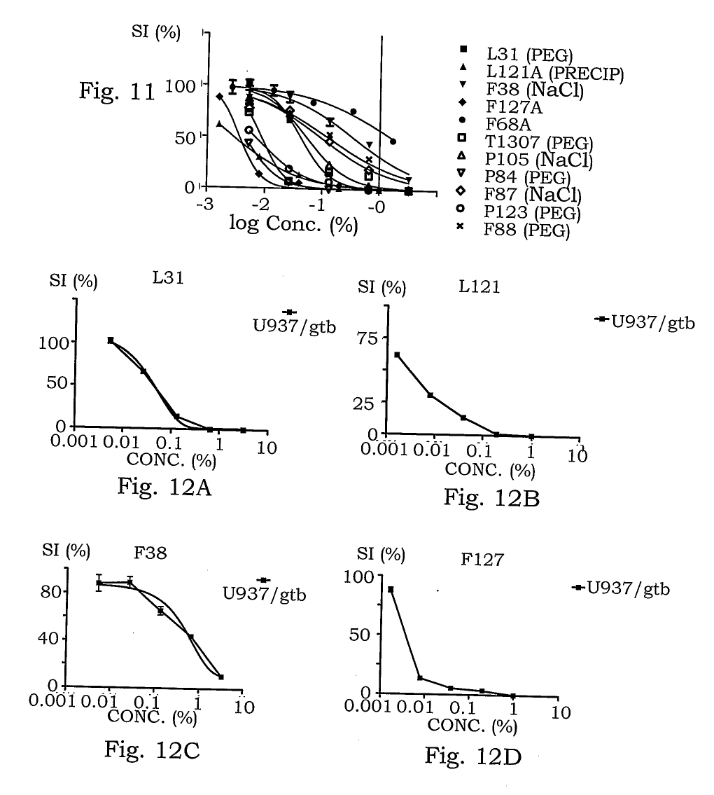

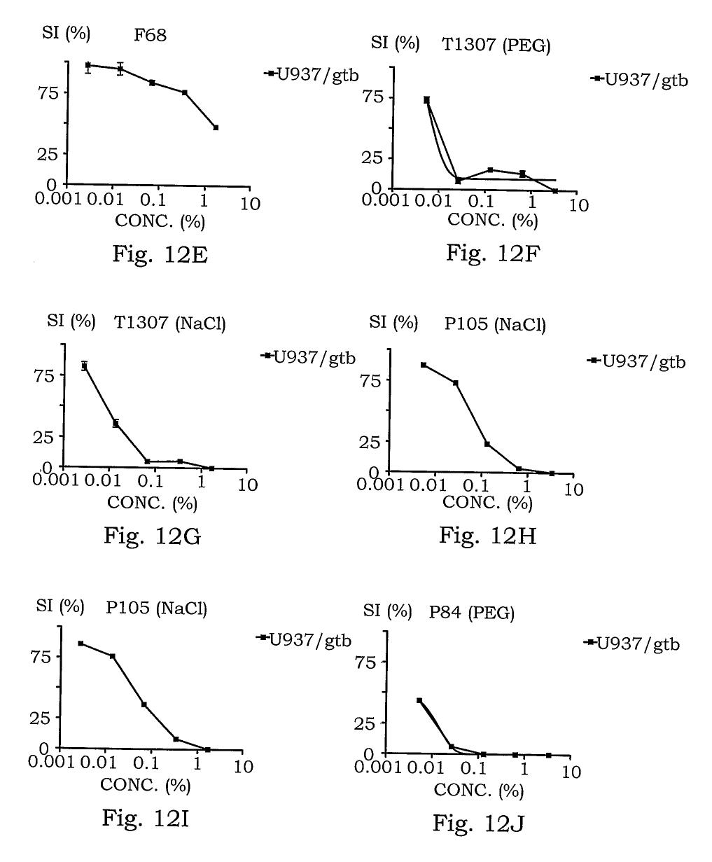

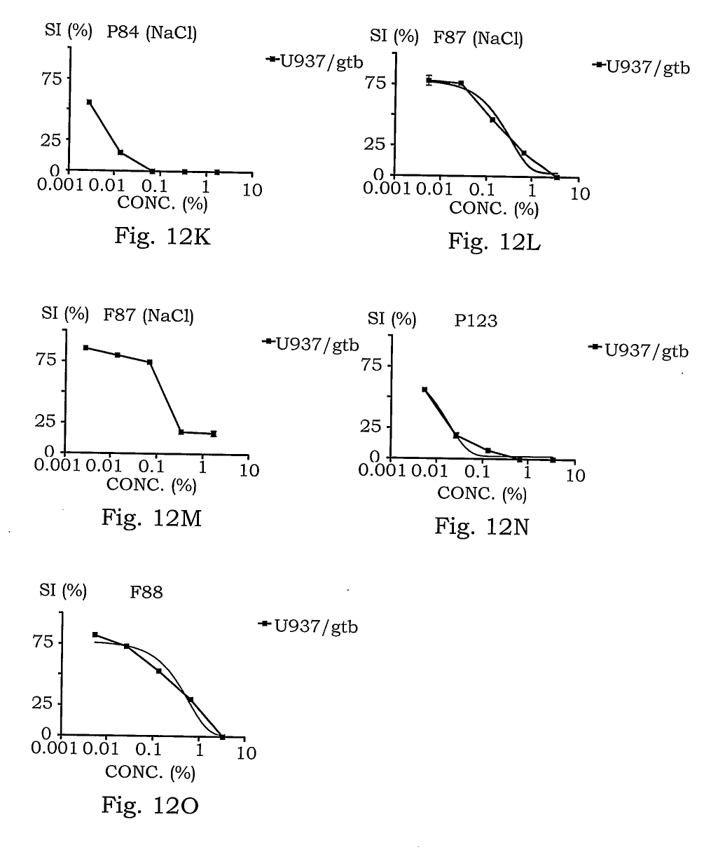

The concentration-effect curves for all tested compounds in U937gtb are shown in FIG. 11. The respective tested copolymers are individually depicted in the FIGS. 12A to 12O. The IC50-values are shown in Table X below. When samples dissolved in NaCl/EtOH and PEG/NaCl/EtOH were compared similar results were obtained (not shown).

TABLE X
IC50 values for copolymers

| Copolymer | IC50 (% w/w) |
|---|---|
| L31 | 0.042 |
| T1307 | 0.0085 |
| F38 | 0.35 |
| P84 | 0.045 |
| P105 | 0.053 |
| L121* | 0.0029 |
| F68 | 1.7 |
| F127 | 0.0037 |
| F87 | 0.094 |
| P123 | 0.0067 |
| F88 | 0.13 |

*L121 precipitated upon dilution in PBS to yield a milky suspension, considered adequately homogenous for testing.

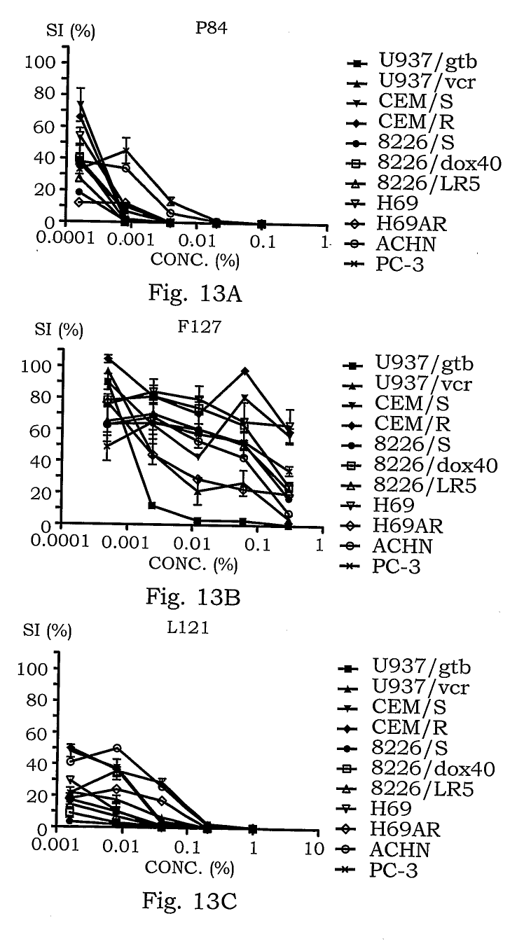

Once again, the results confirm that PLURONIC® copolymer with an average hydrophilic content of about 80% has by far the lowest anti-cancer effect. The EC50 for all cell lines are presented in Table XI for the three most effective copolymers selected from Table XI. FIGS. 13A to 13C are graphical presentations of the results.

TABLE XI
EC 50 Activity in the cell line panel

| Cell line | F127 (% w/w) | P84 (% w/w) | L121 (% w/w) |
|---|---|---|---|
| CCRF-CEM | >1 | 0.022 | 0.0018 |
| CEM/VM-1 | >1 | 0.0020 | 0.0020 |
| ACHN | 0.0095 | 0.00091* | 0.0019 |
| NCI-H69 | >1 | 0.0018 | 0.00061* |
| H69AR | 0.0028 | 0.00003* | <<0.0016* |
| RPMI 8226/S | 0.017 | 0.00075* | <<0.0016* |
| 8226/dox40 | 0.078 | 0.0014 | <<0.0016* |
| 8226/LR5 | 0.024 | 0.0015 | 0.00012* |
| U-937 GTP | 0.0011 | 0.0012 | 0.00012* |
| U-937-vcr | 0.0032 | 0.0011 | 0.00012* |
| PC-3 | 0.027 | 0.00076* | <<0.0016* |

Approximated value, curve-fitting not possible.

The substances were tested down to a minimum concentration of 0.0016% w/w. EC50-values below this are estimations from the linear regression analyses allowing extrapolation of the curve. When curve fitting was inadequate and a majority of cells were dead at the lowest concentration EC50<<0.0016 was used.

It will be understood by a person skilled in the art that various modifications and changes may be made to the present invention without departure from the scope thereof, which is defined by the appended claims.

### REFERENCES

[1] US 2001/0051659 A1
[2] Silk et al., Cancer 1972, 29, 171-172
[3] Dhar et al., Br J Cancer 1996, 74, 888-896
[4] Alley et al., Cancer Res 1988, 48, 589-601
[5] Paull et al., J Natl Cancer Inst 1989, 81, 1088-1092
[6] Dhar et al., Eur J Pharmacol 1998, 346, 315-322
[7] Fisher et al., Eur J Cancer 1996, 32A, 1082-1088
[8] Fridborg et al., Eur J Cancer 1999, 35, 424-432
[9] Valeriote et al., Cancer Chemother Rep 1975, 59, 895-900
[10] Dalton et al., Cancer Res 1986, 46, 5125-5130
[11] Bellamy et al., Cancer Res 1991, 51, 995-1002
[12] Botling et al., Int J Cancer 1994, 58, 269-274
[13] Cole et al., Science 1992, 258, 1650-1654
[14] Danks et al., Biochemistry 1988, 27, 8861-8869
[15] Nygren et al., Biosci Rep 1990, 10, 231-237
[16] Larsson et al., Int J Cancer 1992, 50, 177-185
[17] Freireich et al., Cancer Chemother Rep 1966, 50, 219-244
[18] WO 98/07434
[19] Kabanov et al., Advanced Drug Delivery Reviews 2002, 54, 759-779

1-20. (canceled)

21. An amphiphilic block copolymer having a central hydrophobic polymer chain with a first end connected to at least one hydrophilic ethylene oxide side polymer chain and a second end connected to at least one hydrophilic ethylene oxide side polymer chain as chemotherapeutic agent in the manufacture of a medicament for treating or preventing cancer with the proviso that said cancer is not colon or rectal cancer, wherein an average content of said ethylene oxide constitutes less than 80% of said amphiphilic block copolymer and said medicament comprises said amphiphilic block copolymer or a mixture of at least two amphiphilic block copolymers as the sole chemotherapeutic agent.

22. An amphiphilic block copolymer having a central hydrophobic polymer chain with a first end connected to at least one hydrophilic ethylene oxide side polymer chain and a second end connected to at least one hydrophilic ethylene oxide side polymer chain in the manufacture of a medicament for reducing a growth rate of cancer cells in a subject suffering from cancer with the proviso that said cancer is not colon or rectal cancer, wherein an average content of said ethylene oxide constitutes less than 80% of said amphiphilic block copolymer and said medicament comprises said amphiphilic block copolymer or a mixture of at least two amphiphilic block copolymers as the sole growth rate reducing agent.

23. An amphiphilic block copolymer having a central hydrophobic polymer chain with a first end connected to at least one hydrophilic ethylene oxide side polymer chain and a second end connected to at least one hydrophilic ethylene oxide side polymer chain in the manufacture of an anticell-proliferation medicament with the proviso that said cell is not a colon or rectal cancer cell, wherein an average content of said ethylene oxide constitutes less than 80% of said amphiphilic block copolymer and said medicament comprises said amphiphilic block copolymer or a mixture of at least two amphiphilic block copolymers as the sole anti-cell-proliferation agent.

24. The amphiphilic block copolymer according to claim 21, wherein said amphiphilic block copolymer is represented by formula (I):
HO—(CH₂CH₂O)ₙ—(CH(CH₃)CH₂O)ₘ—(CH₂CH₂O)ₚ—H (I)
where m, n and p are each integer numbers.

25. The use according to claim 24, wherein m, n and p are selected so that
$$\frac{44 \times ( n + p )}{44 \times ( n + p ) + 58 \times m} < 0.8 .$$

26. The amphiphilic block copolymer according to claim 24, wherein n is equal to p.

27. The amphiphilic block copolymer according to claim 21, wherein an average ethylene oxide content of said amphiphilic block copolymer is at least 40% w/w but below 80% w/w.

28. The amphiphilic block copolymer according to claim 24, wherein an average propylene oxide content of said amphiphilic block copolymer is at least 2 000 g/mol.

29. The amphiphilic block copolymer according to claim 28, wherein said average propylene oxide content is at least 3 000 g/mol.

30. The amphiphilic block copolymer according to claim 29, wherein said average propylene oxide content is in a range of 3 500 to 4 500 g/mol, preferably about 4 000 g/mol.

31. The amphiphilic block copolymer according to claim 21, wherein said amphiphilic block copolymer has an average molecule weight of 12 600 g/mol, an average ethylene oxide content of 73.2±1.7% and a melting point of 56° C.

32. The amphiphilic block copolymer according to claim 31, wherein said cancer is selected from the group consisting of renal cancer, lung cancer, myeloma, lymphoma and prostate cancer.

33. An in vitro method of modulating a proliferation rate of a cell with the proviso that said cell is not colon or rectal cancer cell, said method comprising contacting said cell with an amphiphilic block copolymer having a central hydrophobic polymer chain with a first end connected to at least one hydrophilic ethylene oxide side polymer chain and a second end connected to at least one hydrophilic ethylene oxide side polymer chain, wherein an average content of said ethylene oxide constitutes less than 80% of said amphiphilic block copolymer.

34. The method according to claim 33, wherein said cell is a cancer cell but not a colon or rectal cancer cell.

35. A method of blocking binding of a growth factor to a growth factor receptor on a cell membrane of a cell, said method comprising contacting said cell with an amphiphilic block copolymer having a central hydrophobic polymer chain with a first end connected to at least one hydrophilic ethylene oxide side polymer chain and a second end connected to at least one hydrophilic ethylene oxide side polymer chain, wherein an average content of said ethylene oxide constitutes less than 80% of said amphiphilic block copolymer.

36. The method according to claim 35, wherein said growth factor is a fibroblast growth factor or a platelet derived growth factor.

37. A method of treating or preventing cancer in a subject, with the proviso that said cancer is not colon or rectal cancer, said method comprising administering, to said subject, a medicament comprising an amphiphilic block copolymer having a central hydrophobic polymer chain with a first end connected to at least one hydrophilic ethylene oxide side polymer chain and a second end connected to at least one hydrophilic ethylene oxide side polymer chain as chemotherapeutic agent, wherein an average content of said ethylene oxide constitutes less than 80% of said amphiphilic block copolymer and said medicament comprises said amphiphilic block copolymer or a mixture of at least two amphiphilic block copolymers as the sole chemotherapeutic agent.

38. A method of reducing a growth rate of cancer cells in a subject suffering from cancer with the proviso that said cancer is not colon or rectal cancer, said method comprising administering, to said subject, a medicament comprising an amphiphilic block copolymer having a central hydrophobic polymer chain with a first end connected to at least one hydrophilic ethylene oxide side polymer chain and a second end connected to at least one hydrophilic ethylene oxide side polymer chain, wherein an average content of said ethylene oxide constitutes less than 80% of said amphiphilic block copolymer and said medicament comprises said amphiphilic block copolymer or a mixture of at least two amphiphilic block copolymers as the sole growth rate reducing agent.

39. A method inhibiting cell proliferation in a subject, with the proviso that said cell is not a colon or rectal cancer cell, said method comprising administering, to said subject, an anti-cell-proliferation medicament comprising an amphiphilic block copolymer having a central hydrophobic polymer chain with a first end connected to at least one hydrophilic ethylene oxide side polymer chain and a second end connected to at least one hydrophilic ethylene oxide side polymer chain, wherein an average content of said ethylene oxide constitutes less than 80% of said amphiphilic block copolymer and said medicament comprises said amphiphilic block copolymer or a mixture of at least two amphiphilic block copolymers as the sole anticell-proliferation agent.
